(19) United States
(12) Patent Application Publication
(10) Pub. No.: US 2006/0068012 A1
(43) Pub. Date: Mar. 30, 2006

(54) Process for preparing poly (vinyl alcohol) drug delivery devices with humidity control

(75) Inventors: David Joseph Heiler, Avon, NY (US); Susan P. Spooner, Cary, NC (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/229,953

(22) Filed: Sep. 19, 2005

Related U.S. Application Data
(60) Provisional application No. 60/614,370, filed on Sep. 29, 2004.

(51) Int. Cl. A61K 9/28; A61K 9/24; B05D 3/00

(52) U.S. Cl. 424/473; 427/2.14

(57) ABSTRACT

The present invention is a process for making a plurality of drug delivery devices for ocular delivery. The plurality of drug delivery devices are made in part of poly(vinyl alcohol). During the manufacturing process the poly(vinyl alcohol) is cured. The poly(vinyl alcohol) may be in the form of separate pieces, a unitary sheet or may be incorporated into the drug delivery device at the time of curing. During the step of curing the humidity is controlled to ensure improved consistency during the curing process. The improved consistency results in inventories of drug delivery devices that have different cure times.

### CROSS REFERENCE

This application claims the benefit of Provisional Patent Application No. 60/614,370 filed Sep. 29, 2004 and is incorporated herein by reference.

### BACKGROUND OF THE INVENTION

1. Field of the Invention

This invention relates generally to the field of drug delivery devices, and more particularly to the field of drug delivery devices that are placed or implanted into the eye or ocular region of a patient to release a therapeutically active agent to the eye or ocular region of a patient.

2. Description of Related Art

Various drugs have been developed to assist in the treatment of a wide variety of ailments and diseases. However, in many instances, such drugs cannot be effectively administered orally or intravenously without the risk of detrimental side effects. Additionally, it is often desired to administer a drug locally, i.e., to the area of the body requiring treatment. Further, it may be desired to administer a drug locally in a sustained release manner, so that relatively small doses of the drug are exposed to the area of the body requiring treatment over an extended period of time.

Drug delivery to the eye of a patient is further desirable because very little of a therapeutically active agent that is administered systemically typically passes the blood/retinal barrier. Injection of a therapeutically active agent into the eye of a patient in the form of a bolus injection is undesirable because it often requires repeated injections, particularly when the condition to be treated requires administration over a long-term period.

Accordingly, various sustained release drug delivery devices have been proposed for placing in the eye and treating various eye diseases. Examples are found in the following patents, the disclosures of which are incorporated herein by reference: U.S. 2002/0086051A1 (Viscasillas); U.S. 2002/0106395A1 (Brubaker); U.S. 2002/0110591A1 (Brubaker et al.); U.S. 2002/0110592A1 (Brubaker et al.); U.S. 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); U.S. Pat. No. 6,375,972 (Guo et al.); U.S. patent application Ser. No. 10/403,421 (Drug Delivery Device, filed Mar. 28, 2003) (Mosack et al.); and U.S. patent application Ser. No. 10/610,063 (Drug Delivery Device, filed Jun. 30, 2003) (Mosack). Poly(vinyl alcohol) and other materials that are permeable to the active agent require heat curing.

Many of these devices include at least one layer of material permeable to the active agent, such as poly(vinyl alcohol). The poly(vinyl alcohol) is believed to control the rate of release of the therapeutically active agent from the drug delivery device. It is important to have a process for mass-producing drug delivery devices by batch or continuous processes in such a way that will result in drug delivery devices with a greater consistency of drug release profiles.

U.S. Pat. No. 5,378,475 discloses an ophthalmic drug delivery device that was made by a first and second coating layer. The first coating layer is ethylene vinyl acetate. The second coating layer is poly(vinyl alcohol). It was taught that after the second coating was applied. The device was heated to adjust the permeability of the outer coating.

In an article by Nikolas Peppas, entitled “Kinetics of the Crystalization of Cross-linked Poly(vinyl alcohol) Films by Slow Evaporation of Hydrogels,” p. 469-479. concluded that the degree of crystallinity wx is a function of time and the rate of dehydration rp. The rate of dehydration was believed to be affected by the drying condition of the hydrogels that were studied. Temperature, relative humidity and water content of the samples were believed to be factors affecting cross-linking.

There is still a need for a process for manufacturing a plurality of drug delivery devices that use poly(vinyl alcohol) to control the rate of release of the therapeutically active agent from the drug delivery devices in a way that improves the consistency of the release profile from one drug delivery device to another. The present invention addresses this and other needs.

### SUMMARY OF THE INVENTION

The present invention is a process for making a plurality of drug delivery devices for implantation in the eye of a patient. The devices have greater consistency from one device to the next because variations in humidity are controlled during the curing process. “Curing” is defined as the non-chemical, cross-linking of PVA by crystallization.

In one embodiment, the process comprises providing a first drug delivery device and a second drug delivery device. The first drug delivery device and the second drug delivery device each comprise a therapeutically active agent and uncured poly(vinyl alcohol). Furthermore, the first drug delivery device and the second drug delivery device are sized and configured to be inserted into the eye of a patient.

Additionally, the poly(vinyl alcohol) in the first drug delivery device and the second drug delivery device are cured. The poly(vinyl alcohol) in the first drug delivery device and the second drug delivery device are separated by a predetermined distance for a time period. The humidity proximate the first drug delivery device and the humidity proximate the second drug delivery device varies by a maximum of 30% points relative humidity. In one embodiment, the cured poly(vinyl alcohol) in the first drug delivery device and the second drug delivery device are located relative to the therapeutically active agent in each of the first drug delivery device and the second drug delivery device to effect the rate of release of the therapeutically active agent from the drug delivery device.

In another embodiment, there is a process for making a drug delivery device for implantation in the eye of a patient. The process comprises providing a therapeutically active agent in a first part and a second part. Additionally, uncured poly(vinyl alcohol) is provided in a first portion and a second portion. The first portion of poly(vinyl alcohol) and second portion of poly(vinyl alcohol) are cured. During curing the first portion of poly(vinyl alcohol) and the second portion of poly(vinyl alcohol) are separated by a predetermined distance for a time period, wherein the humidity proximate the first portion and the second portion varies by a maximum of 30% points relative humidity. The first portion of poly(vinyl alcohol) and a second portion of poly(vinyl alcohol) are combined with the respective first part and the second part in a respective first drug delivery device and a second drug delivery device.

In one embodiment, the first portion of poly(vinyl alcohol) and the second portion of poly(vinyl alcohol) are located relative to the respective first part and the second part in the respective first drug delivery device and the second drug delivery device to effect the rate of release of the first part and the second part from the drug delivery device. Typically, the first portion is mixed with the first part to form a matrix and the second portion is mixed with the second part to form a second matrix. Optionally, the first part and the second part is formed into respective first drug core and second drug core and the first portion encapsulates at least a portion of the first drug core and the second portion encapsulates at least a portion of the second drug core.

In another embodiment, the first portion and the second portion encapsulates the entire first drug core and the entire second drug core, respectively.

In still another embodiment, the first drug core and the second drug core are at least partly covered with an impermeable polymer material. Optionally, the first portion and second portion form an inner covering and the impermeable polymer material form an outer coating. Typically, the step of combining occurs after the step of curing. Alternatively, the step of combining occurs before the step of curing.

In another embodiment, the step of providing further comprises providing a respective first drug core from the first portion and a second drug core from the second portion and further define providing a respective first cup and second cup that are impermeable to the passage of the therapeutically active agent and define respective first internal compartment and second internal compartment that are sized and configured to receive the first drug core and the second drug core respectively. The first unitary cup and the second unitary cup each define respective first opening and second opening. The step of providing a portion provides a respective first cover made from the first portion and second cover made from the second portion. The step of combining further comprises placing the first cover in a covering relationship to the first opening and placing the second cover in a covering relationship to the second opening.

In one embodiment, the step of combining occurs after the step of curing. In another embodiment, the first portion and the second portion, when cured, form a barrier through which the therapeutically active agent in each of the respective first drug delivery device and second drug delivery device passes into the eye of the patient. In another embodiment, the first portion and the second portion, when cured, are positioned relative to the therapeutically active agent in each of the first drug delivery device and the second drug delivery device to effect the rate of release of therapeutically active agent from each of the first drug delivery device and second drug delivery device.

In still another embodiment, there is a process for making a plurality of drug delivery devices for implantation in the eye of a patient. The process comprises the step of providing a plurality of amounts of therapeutically active agent. Then, a plurality of portions of poly(vinyl alcohol) are provided. The plurality of portions are separated by a predetermined distance for a time period, and the humidity proximate any one of the plurality of portions vary from any other of the plurality of portions by a maximum of 30% points relative humidity. Additionally, each of the plurality of amounts of therapeutically active agent are combined with each of the plurality of portions of poly(vinyl alcohol) to form a drug delivery device.

In one embodiment, wherein each of the plurality of portions of poly(vinyl alcohol) are located relative to each of corresponding plurality of amounts of therapeutically active agent in each of corresponding plurality of drug delivery devices to effect the rate of release of therapeutically active agent from each of the plurality of drug delivery devices. In another embodiment, each of the plurality of portions are mixed with each of corresponding plurality of amounts to form a corresponding plurality of matrices.

In still another embodiment, the plurality of amounts are formed into a plurality of drug cores and the corresponding plurality of portions cover at least a portion of each of the plurality of drug cores. The plurality of portions each cover corresponding plurality of drug cores. The plurality of cores are in part covered with an impermeable polymer material. The plurality of portions form an inner covering over corresponding plurality of drug cores and the impermeable polymer material form an outer coating on each of the plurality of drug cores. In an embodiment, the step of combining occurs after the step of curing. Alternatively, the step of combining occurs before the step of curing.

In an embodiment, the step of providing a plurality of amounts comprises providing a plurality of drug cores from the plurality of amounts, the plurality of drug cores are placed inside of a corresponding plurality of compartments that are defined by a plurality of cups, the plurality of cups are impermeable to the passage of the therapeutically active agent, the plurality of cups further define a corresponding plurality of openings. The step of providing a plurality of portions comprises providing a plurality of covers made from the plurality of portions. The step of combining comprises placing the plurality of covers in a covering relationship to the first opening and placing the second cover in a covering relationship to the second opening.

The present invention also includes an inventory of drug delivery devices manufactured according to one or more embodiments of the present invention. The inventory is unique in that poly(vinyl alcohol) portion are cured more consistently from one drug delivery device to the next.

### BRIEF DESCRIPTION OF THE DRAWING

FIG. 1 of the present invention is an enlarged cross-sectional view down the center of one embodiment of the sustained release drug delivery device.

FIG. 2 is a cross sectional view of a first embodiment of a drug delivery device of this invention.

FIG. 3 is a second cross-sectional view of the device of FIG. 1 viewed along the line 3-3.

### DETAILED DESCRIPTION OF THE INVENTION

The present invention is a process for making a plurality of drug delivery devices for implantation in the eye of a patient that are made in part of poly(vinyl alcohol). The poly(vinyl alcohol) is cured more consistently from one device to the next because, the process includes maintaining the oven at a more consistent humidity. The consistent curing results in less variation in the drug release rate from one device to the next. Any improvement in the consistency of the release rate is of significant benefit. Inventories of devices with greater consistency are more valuable to the physician because, the physician can rely with a greater degree of confidence on the delivery profile.

The process comprises the step of providing a plurality of drug delivery devices preferably more than about 100, about 200, about 400 or about 1000 devices—preferably about 1400 devices.

The devices according to one embodiment include drug reservoir devices where a therapeutically active agent forms a drug core. The drug core is encapsulated, at least in part, with poly(vinyl alcohol). In another embodiment, the drug core is housed within a housing that is made at least in part by polyvinyl alcohol. In still another embodiment, the poly(vinyl alcohol) is coated, at least in part, onto the surface of the drug core. In another embodiment, the therapeutically active agent forms a drug core and the first drug delivery device and second drug delivery device comprises a poly(vinyl alcohol) covering that covers at least a portion of the therapeutically active agent. In one embodiment, the poly(vinyl alcohol) covering covers the entire drug core. In another embodiment, the cured poly(vinyl alcohol) in the drug delivery device form a barrier through which the therapeutically active agent in the drug delivery device passes into the eye of the patient.

The size and thickness of the permeable membrane determines the rate of diffusion and delivery of the medicament. The permeable membrane can be a coating applied directly to the surface of all or a portion of the surface of the therapeutically active agent or all or part of a preformed hosing that surrounds the therapeutically active agent. Examples of devices with permeable membranes are found in U.S. 2002/0086051A1 (Viscasillas); U.S. 2002/0106395A1 (Brubaker); U.S. 2002/0110591A1 (Brubaker et al.); U.S. 2002/0110592A1 (Brubaker et al.); U.S. 2002/0110635A1 (Brubaker et al.); U.S. Pat. No. 5,378,475 (Smith et al.); U.S. Pat. No. 5,773,019 (Ashton et al.); U.S. Pat. No. 5,902,598 (Chen et al.); U.S. Pat. No. 6,001,386 (Ashton et al.); U.S. Pat. No. 6,217,895 (Guo et al.); U.S. Pat. No. 6,375,972 (Guo et al.); U.S. patent application Ser. No. 10/403,421 (Drug Delivery Device, filed Mar. 28, 2003) (Mosack et al.); and U.S. patent application Ser. No. 10/610,063 (Drug Delivery Device, filed Jun. 30, 2003) (Mosack) all of which are incorporated by reference.

In one embodiment, the poly(vinyl alcohol) in the first drug delivery device and the second drug delivery device form a barrier through which the therapeutically active agent in each of the first drug delivery device and the second drug delivery device passes into the eye of the patient. In another embodiment, the poly(vinyl alcohol) in the first drug delivery device and the second drug delivery device are positioned relative to the therapeutically active agent in each of the first drug delivery device and the second drug delivery device to effect the rate of release of therapeutically active agent from each of the first drug delivery device and second drug delivery device.

In another embodiment, the drug reservoir device is made at least in part of an impermeable polymer material. The permeable polymer material covers, houses, coats or encapsulates at least a portion of the drug core.

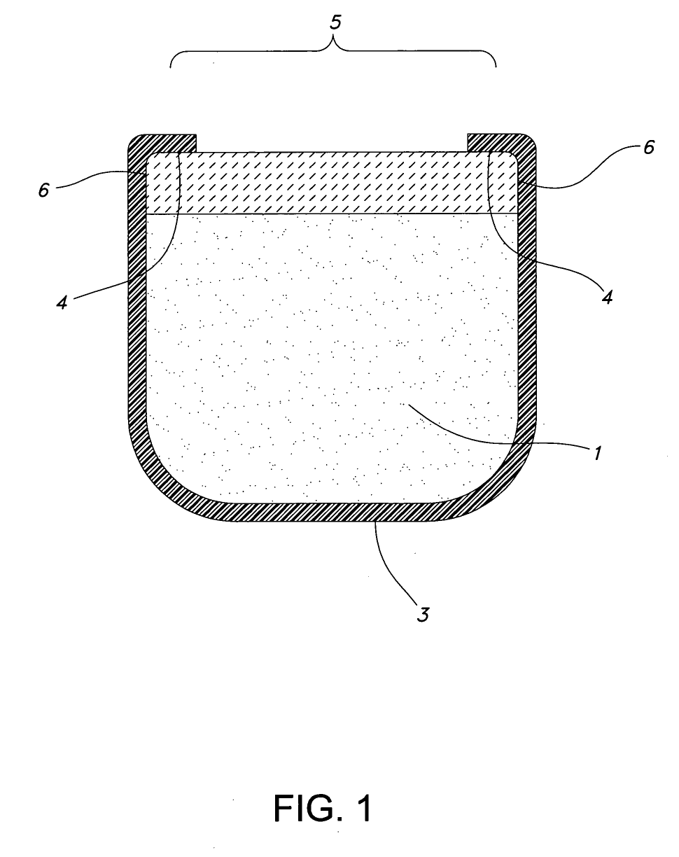

Without limiting the invention to a particular embodiment, FIG. 1 illustrates one type of drug reservoir device. The reservoir is defined by a U-shaped cup 3 that is made of an impermeable material and contains a drug core 1. The drug core is made at least in part made of a therapeutically active agent. The cup 3 has one or more lips 4 extending inward around the open top end 5 of the cup 3. A prefabricated plug 2 formed of poly(vinyl alcohol) is positioned in the recess between the top end of the drug core 1 and below the one or more lips 4 such that the one or more lips 4 interact with the prefabricated plug 2 holding it in position and closing the open top end 5 of the cup 3.

The one or more lips 4 are made of the the same impermeable material as the unitary cup 3 and protrude inwardly from the top open end 5 of the cup 3. In one embodiment, the cup 3 and lips 4 are formed in a single unitary design to provide structural integrity to the device and facilitate manufacturing and handling. The lips 4 are designed to enable the prefabricated plug 2 to snap into place and then to hold the plug 2 in place during use. They can vary in size or shape. The lips 4 of the present invention include nubs, tabs, ridges, and any other raised or protruding member.

By prefabricating the permeable plug 2 it can be snapped into or securely placed in the device in one step. The prefabricated plug 2 can be fabricated or machined to various dimensional specifications, which can be used to control diffusion properties to achieve a desired release rate. The same unitary cup and lips design can be used for implants with a variety of release rates making it possible to use a single manufacturing line or type of equipment. Thus, the present invention allows for ease of construction by more standard manufacturing techniques into devices with different release rates.

Together the cup 3 with lips 4 and the prefabricated permeable plug 2 acts as a reservoir surrounding the drug core 1 to keep the drug core in place. The therapeutically active agent diffuses out of the drug core 1, through the prefabricated permeable plug 2, and out of the open top end 5. The prefabricated plug 2 has substantially the same radial extent as the cup 3, so that the only diffusion pathway is out of the plug 2 and not around the sides 6. Glue, a polymeric substance or other adhesion means can be employed to further bond the plug to the cup.

For one embodiment, the therapeutically active agent may be provided in the form of a micronized powder, and then mixed with an aqueous solution of poly(vinyl alcohol), whereby the active agent and poly(vinyl alcohol) agglomerate into larger sized particles. The resulting mixture is then dried to remove some of the moisture, and then milled and sieved to reduce the particle size so that the mixture is more flowable. Optionally, a small amount of inert lubricant, for example, magnesium stearate, may be added to assist in tablet making. This mixture is then formed into a tablet using standard tablet making apparatus, this tablet representing inner drug core 1.

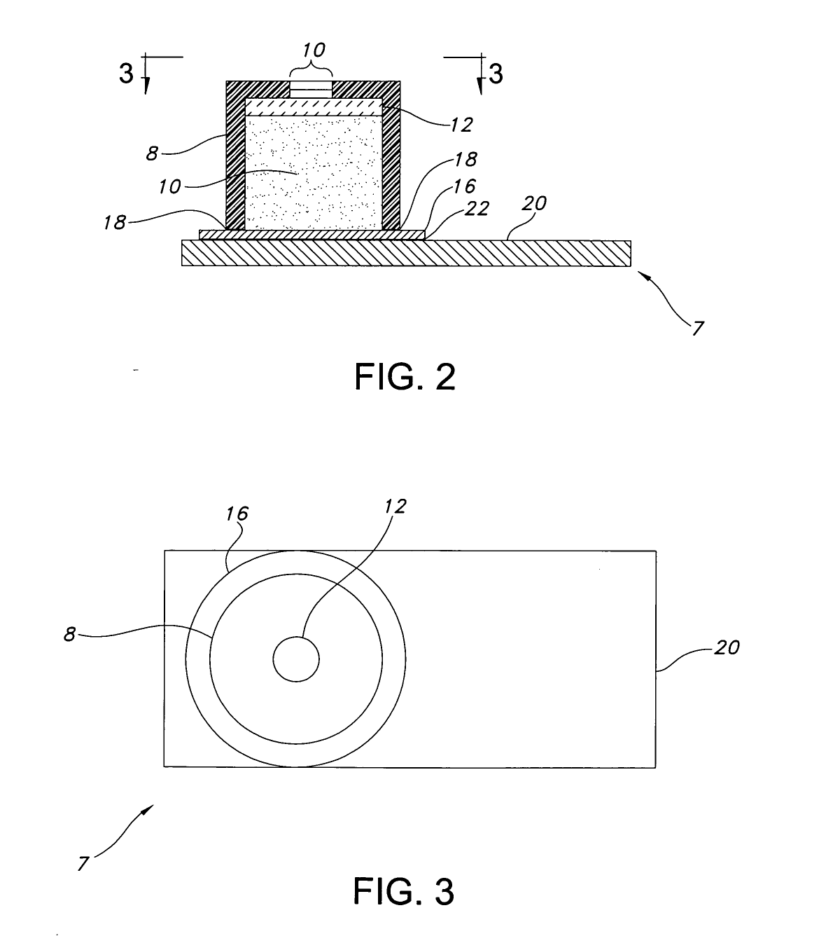

Another device is made according to one embodiment of the present invention is described with reference to FIGS. 2 and 3. Device 7 is a sustained release drug delivery device for implanting in the eye. Device 7 includes inner drug core 10 including a therapeutically active agent.

As shown in FIGS. 2 and 3, therapeutically active agent, optionally, is mixed with a matrix material to effectively bind the therapeutically active agent into a tablet form for easy insertion into the drug delivery device 7. Preferably, the matrix material is a polymeric material that is compatible with body fluids and the eye. Additionally, matrix material should be permeable to passage of the therapeutically active agent therethrough, particularly when the device is exposed to body fluids. In one embodiment, the matrix material is PVA. Also, in one embodiment, the therapeutically active agent is optionally coated with a coating permeable polymeric material, which is the same or different from material mixed with the therapeutically active agent.

The drug delivery device 7 includes a cup shape holder 8 for the inner drug core 10. Holder 8 is made of a material that is impermeable to passage of the therapeutically active agent. Since holder 8 is made of the impermeable material, an opening 14 is formed in holder 8 to permit the therapeutically active agent to pass through the opening 14 and contact the surrounding eye tissue. A drug permeable membrane 12 is positioned in the holder 8 between the drug core 10 and the opening 14 to further effect the rate of release of the therapeutically active agent from the drug delivery device 7.

The cup shaped holder 8, of one embodiment, has a mouth portion opposite the opening 14 that is configured to receive the prefabricated permeable membrane 12 and the drug core 10 during manufacture. A sealable lid 16 is placed over the mouth of the holder and sealed by an adhesive layer 18. The sealable lid 16, of one embodiment, is made of an impermeable material such as silicone. The sealable lid 16 optionally has an extended portion such as a suture tab (not shown in this form) that is configured to suture the drug delivery device to adjacent tissue in the patient's eye according to techniques that are recognized in the art.

In the illustrated embodiment, a suture tab 20 is affixed to the lid 16 by an adhesive layer 22. The suture tab 20, illustrated in the present invention, is made of polyvinyl alcohol.

In one embodiment the preformed disk made of poly(vinyl alcohol). In assembling this embodiment, a solution of uncured poly(vinyl alcohol) is distributed evenly and dried into uncured poly(vinyl alcohol) sheets. The sheets are placed into an oven or drier for curing.

The drug delivery device of FIGS. 2 and 3 is made by providing an impermeable cup shaped holder 8. The cup shape holder 8 has an opening 14 that is sized and configured to effect the rate of release of the therapeutically active agent from the drug delivery device. The holder 8 also has a mouth. Typically, the mouth is larger than the opening 14. A drug permeable membrane 12 is inserted through the mouth into the cup-shaped holder 8 and is positioned in a covering relationship with the opening 14. Typically, the membrane sealably covers the opening 14. Optionally, the membrane is adhered to the holder 8.

Thereafter, the drug core 10 is inserted into the cup shaped holder 8 adjacent the drug permeable membrane. Thereafter, a sealable lid 16 is adhered to the cup shaped holder 8 by an adhesive layer 18. The sealable lid 16 is made of a drug impermeable material such as silicone. Optionally, a suture tab 20 is affixed to the sealable lid 16 by an adhesive layer 22. Suture tab 20 is drug permeable or drug impermeable. In the present invention, the suture tab 20 is made of poly(vinyl alcohol).

The formulation of the implants for use in the invention may vary according to the preferred drug release profile, the particular therapeutically active agent, the condition being treated, and the medical history of the patient.

The implants of the invention are formulated with particles of therapeutically active agent entrapped within a poly(vinyl alcohol) polymer matrix. Release of the therapeutically active agent is achieved by diffusion of entrapped particles of therapeutically active agent and subsequent dissolution and release of agent. Without intending to be limited to a particular mechanism of action. The release kinetics achieved by a matrix of poly(vinyl alcohol) occurs when water is absorbed into the polymer. The therapeutically active agent is released through polymer swelling. The parameters that determine the release kinetics include the size of the drug particles, the ratio of drug to polymer, the surface area exposed, the erosion rate of the polymer, and the method of manufacture.

The implants are preferably monolithic, i.e. having the therapeutically active agent homogenously distributed through the polymer matrix. The poly(vinyl alcohol) is mixed with the therapeutically active agent to form a matrix material. The poly(vinyl alcohol) usually comprises a minimum of about 10 wt. %, about 20 wt. %, about 30 wt. % and/or a maximum of about 80 wt. %, about 70 wt. % or about 60 wt. % of the matrix material. In one embodiment, the poly(vinyl alcohol) is used as a binder for the medicament to assist in forming a tablet. The poly(vinyl alcohol) usually comprises a minimum of about 2 wt. %, about 3 wt. % or 4 wt. % and or a maximum of about 15 wt. %, about 10 wt. %, about 8 wt. % or about 6 wt. % of the final tablet composition.

One of ordinary skill in the art would readily appreciate that the pharmaceutical devices and methods described herein can be prepared and practiced by applying known procedures in the pharmaceutical arts. Thus, the practice of the present invention employs, unless otherwise indicated, conventional techniques of pharmaceutical sciences including pharmaceutical dosage form design, drug development, pharmacology, of organic chemistry, and polymer sciences. See generally, for example, Remington: The Science and Practice of Pharmacy, 19th Ed., Mack Publishing Co., Easton, Pa. (1995) (hereinafter REMINGTON).

The formulation of the implants for use in the invention may vary according to the preferred drug release profile, the particular therapeutically active agent, the condition being treated, and the medical history of the patient.

The implants of the invention are formulated with particles of the therapeutically active agent entrapped within a poly(vinyl alcohol) polymer matrix. Release of the therapeutically active agent is achieved by diffusion of entrapped particles of therapeutically active agent and subsequent dissolution and release of agent. Without intending to be limited to a particular mechanism of action. The release kinetics achieved by a matrix of poly(vinyl alcohol) occurs when water is absorbed into the polymer. The therapeutically active agent is released through polymer swelling. The parameters that determine the release kinetics include the size of the drug particles, the ratio of drug to polymer, the surface area exposed, the erosion rate of the polymer, and the method of manufacture.

The implants are preferably monolithic, i.e. having the therapeutically active agent homogenously distributed through the polymer matrix. The poly(vinyl alcohol) is mixed with the therapeutically active agent to form a matrix material. For such an application, the poly(vinyl alcohol) usually comprises a minimum of about 10 wt. %, about 20 wt. %, about 30 wt. % and/or a maximum of about 80 wt. %, about 70 wt. % or about 60 wt. % of the matrix material. In one embodiment, the poly(vinyl alcohol) is used as a binder for the medicament to assist in forming a tablet. For this application, the poly(vinyl alcohol) usually comprises a minimum of about 2 wt. %, about 3 wt. % or 4 wt. % and or a maximum of about 15 wt. %, about 10 wt. %, about 8 wt. % or about 6 wt. % of the final tablet composition.

The size and form of the matrix-type drug delivery device is typically altered to control the rate of release, period of treatment, and drug concentration at the site of implantation. Larger implants will deliver a proportionately larger dose, but depending on the surface to mass ratio, may have a slower release rate. The implants may be particles, sheets, patches, plaques, films, discs, fibers, microcapsules and the like and may be of any size or shape compatible with the selected site of insertion, as long as the implants have the desired release kinetics. Preferably, the implant to be inserted is formulated as a single particle. Preferably, the implant will not migrate from the insertion site following implantation. The upper limit for the implant size will be determined by factors such as the desired release kinetics, toleration for the implant, size limitations on insertion, ease of handling, etc. The vitreous chamber is able to accommodate relatively large implants of varying geometries, having diameters of 1 to 3 mm. In a preferred embodiment, the implant is a cylindrical pellet (e.g., rod) with dimensions of about 2 mm by 0.75 mm diameter. The implants will also preferably be at least somewhat flexible so as to facilitate both insertion of the implant in the vitreous and accommodation of the implant. The total weight of the implant is preferably about 250-5000 [mgr]g, more preferably about 500-1000 [mgr]g. In one embodiment, the implant is about 500 g. In a particularly preferred embodiment, the implant is about 1000 [mgr]g.

The therapeutically active agent is preferably a minimum of about 10 wt. % or about 50 wt. % based upon the weight of the implant and/or a maximum of about 90 wt. %, about 80 wt. %, about 70 wt. %, or about 60 wt. % based upon the weight of the implant. In one preferred embodiment, the therapeutically active agent comprises about 50 wt. % to of the implant. In another preferred embodiment, the therapeutically active agent comprises about 70% by weight of the implant.

In one embodiment, the implants are preferably a monolithic mixture of the therapeutically active agent and the polymer matrix. Preferably, the poly(vinyl alcohol) will not be fully degraded until the drug load has been released. In one embodiment, the poly(vinyl alcohol) a minimum of about 10 wt. %, 20 wt. %, 30 wt. % or about 40 wt. % based upon the weight of the implant and/or a maximum of about 90 wt. %, about 80 wt. %, about 70 wt. % or about 60 wt. % based upon the weight of the implant. In one preferred embodiment, the therapeutically active agent comprises a minimum of about 10 wt. % of the implant. In another preferred embodiment, the therapeutically active agent comprises about 70% by weight of the implant.

Optionally, additional release modulators such as those described in U.S. Pat. No. 5,869,079, which is herein incorporated by reference in its entirety are included in the implants. The amount of release modulator employed will be dependent on the desired release profile, the activity of the modulator, and on the release profile of the therapeutically active agent in the absence of modulator.

The release kinetics of the drug delivery devices of the invention depend in part on the surface area of the devices. The size and form of the implant can be used to control the rate of release, period of treatment, and concentration of therapeutically active agent at the site of implantation. Larger implants will deliver proportionately larger amounts of therapeutically active agent, but depending on surface area of the matrix exposed, the relative concentration of polymer/therapeutically active agent or solubility of the therapeutically active agent may have a varying release rate. The matrix-type drug delivery device may be particles, sheets, patches, plaques, films, discs, fibers, tacks, plugs, coils, microcapsules and the like and are optionally of any size or shape compatible with the selected site of insertion. Preferably, the implant to be inserted is formulated as a single particle. Preferably, the matrix-type drug delivery device will not migrate from the insertion site following implantation. The upper limit for the size of the matrix-type drug delivery device will be determined by factors such as the desired release kinetics, toleration for the implant, dimensional limitations on insertion, ease of handling, etc. The vitreous chamber is able to accommodate relatively large drug delivery devices of varying geometries, including matrix-type drug delivery devices in the shape of a rod or cylindrical pellet having minimum diameters of about 0.5 mm, about 0.75 mm, about 1 mm or about 2 mm and/or a maximum diameter of about 3 mm or about 2 mm in diameter. The matrix-type drug delivery device will be at least somewhat flexible so as to facilitate both insertion of the drug delivery device in the vitreous and accommodation of the device. The total weight of the matrix-type drug delivery device is a minimum of about 250 μg, about 500 μg or about 1000 μg and/or a maximum of about 5000 μg, 1000 μg or 500 μg. In a particularly preferred embodiment, the implant is about 500 μg or about 1000 μg.

The therapeutically active agent is preferably a minimum of about 10 wt. % or about 50 wt. % based upon the weight of the implant and/or a maximum of about 90 wt. %, about 80 wt. %, about 70 wt. %, or about 60 wt. % based upon the weight of the implant. In one preferred embodiment, the therapeutically active agent comprises about 50 wt. % of the implant. In another preferred embodiment, the therapeutically active agent comprises about 70% by weight of the implant.

In one embodiment, the implants are preferably a monolithic mixture of the therapeutically active agent and the polymer matrix. Preferably, the poly(vinyl alcohol) will not be fully degraded until the drug load has been released. In one embodiment, the poly(vinyl alcohol) a minimum of about 10 wt. %, about 20 wt. %, about 30 wt. % or about 40 wt. % based upon the weight of the implant and/or a maximum of about 90 wt. %, about 80 wt. %, about 70 wt. % or about 60 wt. % based upon the weight of the implant. In one preferred embodiment, the therapeutically active agent comprises a minimum of about 10 wt. % of the implant and/or a maximum of about 95 wt. % of the implant. In another preferred embodiment, the therapeutically active agent comprises a minimum of about 20%, about 30% or about 40% by weight of the implant and/or a maximum of about 92 wt. %, about 90 wt. %, about 88 wt. % or about 80 wt. %.

Typically, the implant to be inserted is formulated as a single particle. Preferably, the matrix-type drug delivery device does not migrate from the insertion site following implantation. The upper limit for the size of the matrix-type drug delivery device will be determined by factors such as the desired release kinetics, toleration for the implant, dimensional limitations on insertion, ease of handling, etc. The vitreous chamber is able to accommodate relatively large drug delivery devices of varying geometries, including matrix-type drug delivery devices in the shape of a rod or cylindrical pellet having minimum diameters of about 0.5 mm, about 0.75 mm, about 1 mm or about 2 mm and/or a maximum diameter of about 3 mm or about 2 mm in diameter.

In one embodiment, the delivery device comprises a therapeutically active agent. The therapeutically active agent of one embodiment is selected from the group comprising anesthetics, analgesics, antibiotics, cell transport/mobility impending agents, antiglaucoma drugs, carbonic anhydrase inhibitors, neuroprotectants, antibacterials, anti-fungal agents, anti-viral agents, protease inhibitors, anti-cytomegalovirus agents, antiallergenics, anti-inflammatories, decongestants, miotics, anti-cholinesterases, mydriatics, sympathomimetics, vasoconstrictors, vasodilators, anticlotting agents, antidiabetic agents, aldose reductase inhibitors, anti-cancer agents, hormones, peptides, nucleic acids, saccharides, lipids, glycolipids, glycoproteins, endocrine hormones, growth hormones, heat shock proteins, immunological response modifiers, cyclosporins, interferons (including [agr], [bgr], and [ggr] interferons), cytokines, antineogenesis agents, anti-neovascularization agents, anti-VEGF agents, proteins, monoclonal antibodies, tumor necrosis factor inhibitors, nulceic acids and mixtures thereof.

In a preferred embodiment, the therapeutically active agent is present in an effective amount to treat glaucoma, proliferative vitreoretinopathy, diabetic retinopathy, uveitis, keratitis, cytomegalovirus retinitis, herpes simplex viral or adenoviral infections.

In another embodiment, the therapeutically active agent is selected from the group comprising of colchicine, vincristine, cytochalasin B, timolol, betaxolol, atenolol, acetazolamide, methazolamide, dichlorphenamide, diamox, nimodipine, tetracycline, chlortetracycline, bacitracin, neomycin, polymyxin, gramicidin, oxytetracycline, chloramphenicol, gentamycin, and erythromycin; antibacterials such as sulfonamides, sulfacetamide, sulfamethizole sulfisoxazole, fluconazole, nitrofurazone, amphotericine B, ketoconazole, trifluorothymidine, acyclovir, ganciclovir, DDI, AZT, foscamet, vidarabine, trifluorouridine, idoxuridine, ribavirin, methapyriline, chlorpheniramine, pyrilamine, prophenpyridamine, hydrocortisone, dexamethasone, fluocinolone, prednisone, prednisolone, methylprednisolone, fluorometholone, betamethasone, triamcinolone, phenylephrine, naphazoline, tetrahydrazoline, pilocarpine, carbachol, di-isopropyl fluorophosphate, phospholine iodine, and demecarium bromide, atropine sulfate, cyclopentolate, homatropine, scopolamine, tropicamide, eucatropine, epinephrine, heparin, antifibrinogen, fibrinolysin, acetohexamide, chlorpropamide, glipizide, glyburide, tolazamide, tolbutamide, insulin, 5-fluorouracil, adriamycin, asparaginase, azacitidine, azathioprine, bleomycin, busulfan, carboplatin, carmustine, chlorambucil, cisplatin, cyclophosphamide, cyclosporine, cytarabine, dacarbazine, dactinomycin, daunorubicin, doxorubicin, estramustine, etoposide, etretinate, filgrastin, floxuridine, fludarabine, fluorouracil, fluoxymesterone, flutamide, goserelin, hydroxyurea, ifosfamide, leuprolide, levamisole, lomustine, nitrogen mustard, melphalan, mercaptopurine, methotrexate, mitomycin, mitotane, pentostatin, pipobroman, plicamycin, procarbazine, sargramostin, streptozocin, tamoxifen, taxol, teniposide, thioguanine, uracil, mustard, vinblastine, vincristine, vindesine, insulin-related growth factor, interleukin-2, tacrolimus, tumor necrosis factor, pentostatin, thymopentin, transforming factor beta-2, erythropoietin, anticlotting activase, brain nerve growth factor (BNGF), celiary nerve growth factor (CNGF), vascular endothelial growth factor (VEGF), thalidomide and mixtures thereof.

In a particular embodiment, the therapeutically active agent is a hydrophobic agent. Preferably, the therapeutically active agent will have a solubility that is a maximum of 90 μg/ml in a buffered saline solution at 25° C. Typically, the therapeutically active agent has a solubility that is a maximum of 80 μg/ml, 70 μg/ml, 60 μg/ml, 50 μg/ml, 40 μg/ml, 30 μg/ml, 20 μg/ml, 10 μg/ml or 5 μg/ml.

In one embodiment, the first drug delivery device and the second drug delivery device are sized and configured to be inserted into the eye of a patient. In one embodiment, the first drug delivery device and the second drug delivery device each have a maximum volume of 26 mm3. Typically, the first drug delivery device and the second drug delivery device each have a maximum volume of 15 mm3, 10 mm3, 4 mm3 or 2 mm3.

In one embodiment, there is a first drug delivery device and the second drug delivery device each has a maximum mass of 50 mg. In one embodiment, the first drug delivery device and the second drug delivery device each has a maximum mass of 25 mg, 15 mg, 10 mg, 5 mg or 1 mg.

In another embodiment, the drug delivery devices containing poly(vinyl alcohol) are cured. Alternatively, the poly(vinyl alcohol) portions of the drug delivery devices are cured before being incorporated into the drug delivery devices. Without limiting the invention to a particular theory of operation, it is presently believed that the curing of poly(vinyl alcohol) affects the permeability of water and/or therapeutically active agent. Thus, controlling the conditions, such as humidity, will improve cross-linking. Thus in one embodiment, the first drug delivery device and the second drug delivery device that are separated by a predetermined distance for a time period. During the step of curing the humidity proximate the first drug delivery device and the humidity proximate the second drug delivery device varies by a maximum of 30% relative humidity, wherein the cured poly(vinyl alcohol) in the first drug delivery device and the second drug delivery device are located relative to the therapeutically active agent in each of the first drug delivery device and the second drug delivery device.

In another application, uncured poly(vinyl alcohol) is provided in a first portion and a second portion. The first portion of poly(vinyl alcohol) and the second portion of poly(vinyl alcohol) in one embodiment are preformed before being assembled or incorporated into respectively a first drug delivery device or a second drug delivery device. Additionally, the first portion and the second portion, in one embodiment, refers to portions of the poly(vinyl alcohol) located at different parts of a unitary piece of poly(vinyl alcohol) or may be in part of a different piece. The first portion and the second portion optionally refers to portions of polyvinyl alcohol that are already assembled or incorporated into a respective first drug delivery device or a second drug delivery device.

The first portion of poly(vinyl alcohol) and second portion of poly(vinyl alcohol) are cured in a manner that the humidity proximate the first portion of poly(vinyl alcohol) and the humidity proximate the second portion of poly(vinyl alcohol) varies by a maximum of 30% points of relative humidity. This occurs by using ovens that control humidity such as those that are well known in the art. Constant humidity throughout the oven is believed to improve the consistency of the cure time—particularly when other factors such as temperature are also consistent.

Alternatively, poly(vinyl alcohol) can be cured using standard ovens with only temperature controls by adopting one or more of the following suggestions. The plurality of portions of polyvinyl alcohol, preferably, should not be placed in locations of an oven where the humidity is likely to vary such as an oven vent or air intake (if any). Furthermore, placing a container of water in the oven wherein the amount of water in the container plus the amount of water in the plurality of portions of poly(vinyl alcohol), when hydrated equal the preferred humidity—preferably when a convection oven is used that circulates the air to eliminate temperature and humidity differences effectively. Batch or continuous process may be used for the present invention.

During the step of curing, the plurality of portions are separated by a predetermined distance for a time period, and the humidity proximate any one of the plurality of portions vary from any other of the plurality of portions by a maximum of 30% points relative humidity. Additionally, each of the plurality of amounts of therapeutically active agent are combined with each of the plurality of portions of poly(vinyl alcohol) to form a drug delivery device. In one embodiment, the humidity proximate the first drug delivery device differs from the humidity proximate the second drug delivery device by a maximum of about 25% points relative humidity. In an embodiment, the humidity proximate the first drug delivery device differs from the humidity proximate the second drug delivery device by a maximum of about 20% points, about 15% points, about 10% points, about 5% points or about 3% points relative humidity.

In another embodiment, the humidity proximate the first drug delivery device and the humidity proximate the second drug delivery device is a maximum of about 10% and a minimum of about 95%. The humidity proximate the first drug delivery device and the humidity proximate the second drug delivery device is a minimum of about 20%, of about 30%, of about 40% or of about 50% and/or a maximum of about 90%, of about 80%, of about 70%, of about 60%, or of about 50%.

Optionally, the temperature proximate the first drug delivery device and the temperature proximate the second drug delivery device is a minimum of about 120° C. and a maximum of about 210° C. In an embodiment, the temperature proximate the first drug delivery device and the temperature proximate the second drug delivery device is a minimum of about 125° C., about 130° C., about 135° C. or about 140° C. and/or a maximum of about 210° C., about 200° C., about 180° C., about 170° C., about 150° C. or about 140° C. The desired temperature for curing the poly(vinyl alcohol) is dependent upon several factors. If a temperature is selected below about 120° C., the poly(vinyl alcohol) will not cure effectively. If the temperature is above about 210° C., decomposition of the poly(vinyl alcohol) will occur. If the poly(vinyl alcohol) is cured in the presence of the medicament, the stability of the medicament must be considered. For example, flucinolone acetonide begins to decompose above a temperature of about 165° C. Thus, it is desired that a temperature for curing poly(vinyl alcohol) in the presence of flucinolone acetonide be in the range of about 120° C. to about 150° C. and preferably about 135° C.

In an embodiment, the temperature proximate the first drug delivery device differs from the temperature proximate the second drug delivery device by a maximum of about 25° C. In an embodiment, the temperature proximate the first drug delivery device differs from the temperature proximate the second drug delivery device by maximum of about 20° C., about 15° C., about 10° C. or about 5° C.

During the step of curing, the predetermined distance is a minimum of about 30 cm. In an embodiment, the predetermined distance is a minimum of about 40 cm, about 50 cm, about 60 cm or about 90 cm. Optionally, the time period is a minimum of about 15 minutes and a maximum of about 24 hours. Typically, the time period is a minimum of about 30 min, about 1 hour, about 1.5 hours, about 2 hours, or about 3 hours and/or a maximum of about 20 hours, about 18 hours, about 16 hours, about 12 hours, about 10 hours, or about 8 hours.

In one embodiment, the cured poly(vinyl alcohol) in the first drug delivery device and the second drug delivery device form a barrier through which the therapeutically active agent in each of the first drug delivery device and the second drug delivery device passes into the eye of the patient.

Typically, the cured poly(vinyl alcohol) in the first drug delivery device and the second drug delivery device are positioned relative to the therapeutically active agent in each of the first drug delivery device and the second drug delivery device to effect the rate of release of therapeutically active agent from each of the first drug delivery device and second drug delivery device. This positioning occurs alternatively before or after the curing process. The poly(vinyl alcohol), optionally, is a preformed plug.

In one embodiment, the rate of release of therapeutically active agent from the first drug delivery device is lower than the second drug delivery device by a maximum of about 50% based upon the rate of release of the second drug delivery device. In one embodiment, the rate of release of therapeutically active agent from the first drug delivery device is lower than the second drug delivery device by a maximum of about 40%, about 30%, about 20% or about 10% based upon the rate of release of the second drug delivery device.

The invention further relates to a method for treating a mammalian organism to obtain a desired local or systemic physiological or pharmacological effect. The method includes administering the sustained release drug delivery device to the mammalian organism and allowing the therapeutically active agent effective in obtaining the desired local or systemic physiological or pharmacological effect to pass through the plug. The term “administering,” as used herein, means positioning, inserting, injecting, implanting, or any other means for exposing the device to a mammalian organism. The route of administration depends on a variety of factors including type of response or treatment, type of agent, and the preferred site of administration. However, the preferred method is to insert the device into the target organ. In one ocular application, the device is inserted through a surgical procedure followed by suturing the device in place.

Typically, the present invention can be used to treat any ocular condition, such as, for example, retinal detachment, occlusions, proliferative retinopathy, diabetic retinopathy, inflammations such as uveitis, choroiditis and retinitis, degenerative disease, vascular diseases and various tumors including neoplasms. Kits for the Administration of the Implants

In another aspect of the invention, kits for treating an inflammation-mediated condition of the eye are provided, comprising: a) a container comprising a bioerodible implant comprising dexamethasone and polylactic acid polyglycolic acid (PLGA) copolymer in a ratio of about 70/30; and b) instructions for use.

Methods of implanting a drug delivery device are well-known in the art, and include surgical means, injection, trocar, etc. The ocular implant devices of the present invention may be implanted at several anatomical regions of the eye. For example, the devices may be placed substantially upon the outer surface of the eye and may be anchored in the conjunctiva or sclera, or episclerally or intrasclerally over an avascular region. The devices may also be implanted substantially within the suprachoroidal space over an avascular region such as the pars plana or a surgically-induced avascular region.

Alternatively, the devices may be implanted in an area in direct communication with the vitreal chamber or vitreous so as to avoid diffusion of the therapeutically active agent into the bloodstream. The devices can also be implanted in the anterior chamber. On the other hand, diffusion of the therapeutically active agent to the desired site may be facilitated by forming holes or tunnels through the layers of the sclera or other tissue which communicate, with the desired site of therapy which lie beneath the device. As a result, the tunnels will lie beneath the implant and serve to substantially direct the flow of the therapeutically active agent from the device to the desired site of therapy. These holes may be formed by surgical procedures which are known in the art or through the application of a permeability enhancing agent described above such as ethanol, oleic acid, isopropyl myristate and the like.

Alternatively, the device may be inserted so as to directly communicate with the vitreal chamber. A hole of suitable size may be made through the sclera to communicate with the base of the vitreous body through the pars plana. The implant is positioned over the hole within the scleral bed and the flap of the trap door is sewn back into place. Such placement of the implant will allow for the ready diffusion of the therapeutically active agent into the vitreous and into the intraocular structure.

The devices can be implanted by using an implanter, the operation of which is described in U.S. Pat. Nos. 3,921,632 and 4,451,254. Surgical procedures, such as those known in the art, may be necessary to position large implants. For example, the implants can be inserted through a sclerotomy into the suprachoroid. In this instance, the sclera is cut to expose the suprachoroid. An implant is then inserted on either side of the incision. Alternatively, a partial-thickness scleral trapdoor can be fashioned over the suprachoroid or an avascular region. An implant is then inserted and the scleral flap is sewn back into place to secure the implant.

In many aspects, the device per se can be implanted. In some aspects, the device can be placed in a “container” which is then implanted. For example, the device can be placed in a “container” such as an artifical lens or a limb first and the artificial lens or limb is then ocularly implanted, for example in the anterior chamber. Thus, the devices of this invention are introduced into a body cavity or area in many different ways.

In order to define the potential drug-release behavior of the devices in vivo, the device may be maintained in a measured volume of a saline solution under “in-sink” conditions. The mixture is maintained at 37° C. and agitated or stirred slowly. The appearance of the dissolved therapeutically active agent as a function of time may be followed spectrophotometrically or by other analytical means. While release may not always be uniform, normally the release will be free of substantial fluctuations from some average value, which allows for a relatively uniform release, usually following a brief initial phase of rapid release of the therapeutically active agent. Additional methods are known in the art.

What is claimed is:

1. A process for making a plurality of drug delivery devices, the process comprising the steps of:
(a) providing a first drug delivery device and a second drug delivery device, wherein the first drug delivery device and the second drug delivery device each comprise a therapeutically active agent and uncured poly(vinyl alcohol), wherein the first drug delivery device and the second drug delivery device are sized and configured to be inserted into the eye of a patient; and
(b) curing the poly(vinyl alcohol) in the first drug delivery device and the second drug delivery device that are separated by a predetermined distance for a time period, wherein the humidity proximate the first drug delivery device and the humidity proximate the second drug delivery device varies by a maximum of 30% points relative humidity.

2. The process of claim 1, wherein the first and second drug delivery device comprises poly(vinyl alcohol) that is mixed with the therapeutically active agent to form a drug matrix.

3. The process of claim 1, wherein the therapeutically active agent forms a drug core and the first drug delivery device and second drug delivery device comprises a poly(vinyl alcohol) covering that covers at least a portion of the therapeutically active agent.

4. The process of claim 3, wherein the poly(vinyl alcohol) covering covers the entire drug core.

5. The process of claim 4, wherein the poly(vinyl alcohol) covering is coated onto the surface of the drug core.

6. The process of claim 3, wherein the first drug delivery device and the second drug delivery device further comprises a second covering comprising an impermeable polymer material that covers at least a portion of the drug core.

7. The process of claim 1, wherein the cured poly(vinyl alcohol) in the first drug delivery device and the second drug delivery device form a barrier through which the therapeutically active agent in each of the first drug delivery device and the second drug delivery device passes into the eye of the patient.

8. The process of claim 1, wherein the cured poly(vinyl alcohol) in the first drug delivery device and the second drug delivery device are positioned relative to the therapeutically active agent in each of the first drug delivery device and the second drug delivery device to effect the rate of release of therapeutically active agent from each of the first drug delivery device and second drug delivery device.

9. The process of claim 1, wherein the rate of release of therapeutically active agent from the first drug delivery device is lower than the second drug delivery device by a maximum of about 50% based upon the rate of release of the second drug delivery device.

10. The process of claim 1, wherein the predetermined distance is a minimum of about 30 cm.

11. The process of claim 1, wherein the time period is a minimum of about 15 minutes and a maximum of about 24 hours.

12. The process of claim 1, wherein the therapeutically active agent is a hydrophobic agent.

13. The process of claim 1, wherein the therapeutically active agent is selected from the group comprising anesthetics, analgesics, antibiotics, cell transport/mobility impending agents, antiglaucoma drugs, carbonic anhydrase inhibitors, neuroprotectants, antibacterials, anti-fungal agents, anti-viral agents, protease inhibitors, anti-cytomegalovirus agents, antiallergenics, anti-inflammatories, decongestants, miotics, anti-cholinesterases, mydriatics, sympathomimetics, vasoconstrictors, vasodilators, anticlotting agents, antidiabetic agents, aldose reductase inhibitors, anti-cancer agents, hormones, peptides, nucleic acids, saccharides, lipids, glycolipids, glycoproteins, endocrine hormones, growth hormones, heat shock proteins, immunological response modifiers, cyclosporins, interferons (including [agr], [bgr], and [ggr] interferons), cytokines, antineogenesis proteins, monoclonal antibodies, tumor necrosis factor inhibitors, nulceic acids and mixtures thereof.

14. The process of claim 1, wherein the therapeutically active agent is present in an effective amount to treat glaucoma, proliferative vitreoretinopathy, diabetic retinopathy, uveitis, keratitis, cytomegalovirus retinitis, herpes simplex viral or adenoviral infections.

15. The process of claim 1, wherein the therapeutically active agent is selected from the group comprising colchicine, vincristine, cytochalasin B, timolol, betaxolol, atenolol, acetazolamide, methazolamide, dichlorphenamide, diamox, nimodipine, tetracycline, chlortetracycline, bacitracin, neomycin, polymyxin, gramicidin, oxytetracycline, chloramphenicol, gentamycin, and erythromycin; antibacterials such as sulfonamides, sulfacetamide, sulfamethizole sulfisoxazole, fluconazole, nitrofurazone, amphotericine B, ketoconazole, trifluorothymidine, acyclovir, ganciclovir, DDI, AZT, foscamet, vidarabine, trifluorouridine, idoxuridine, ribavirin, methapyriline, chlorpheniramine, pyrilamine, prophenpyridamine, hydrocortisone, dexamethasone, fluocinolone, prednisone, prednisolone, methylprednisolone, fluorometholone, betamethasone, triamcinolone, phenylephrine, naphazoline, tetrahydrazoline, pilocarpine, carbachol, di-isopropyl fluorophosphate, phospholine iodine, and demecarium bromide, atropine sulfate, cyclopentolate, homatropine, scopolamine, tropicamide, eucatropine, epinephrine, heparin, antifibrinogen, fibrinolysin, acetohexamide, chlorpropamide, glipizide, glyburide, tolazamide, tolbutamide, insulin, 5-fluorouracil, adriamycin, asparaginase, azacitidine, azathioprine, bleomycin, busulfan, carboplatin, carmustine, chlorambucil, cisplatin, cyclophosphamide, cyclosporine, cytarabine, dacarbazine, dactinomycin, daunorubicin, doxorubicin, estramustine, etoposide, etretinate, filgrastin, floxuridine, fludarabine, fluorouracil, fluoxymesterone, flutamide, goserelin, hydroxyurea, ifosfamide, leuprolide, levamisole, lomustine, nitrogen mustard, melphalan, mercaptopurine, methotrexate, mitomycin, mitotane, pentostatin, pipobroman, plicamycin, procarbazine, sargramostin, streptozocin, tamoxifen, taxol, teniposide, thioguanine, uracil, mustard, vinblastine, vincristine, vindesine, insulin-related growth factor, interleukin-2, tacrolimus, tumor necrosis factor, pentostatin, thymopentin, transforming factor beta-2, erythropoietin, anticlotting activase, brain nerve growth factor (BNGF), celiary nerve growth factor (CNGF), vascular endothelial growth factor (VEGF), thalidomide and mixtures thereof.

16. The process of claim 1, wherein the first drug delivery device and the second drug delivery device each have a maximum volume of 26 mm³.

17. The process of claim 1, wherein the first drug delivery device and the second drug delivery device each has a maximum mass of 50 mg.

18. The process of claim 1, wherein the therapeutically active agent comprises a minimum of about 10 wt. % and a maximum of about 95 wt. % of the total mass of the first drug delivery device and the second drug delivery device.

19. The process of claim 1, wherein the humidity proximate the first drug delivery device differs from the humidity proximate the second drug delivery device by a maximum of about 30% points relative humidity.

20. The process of claim 1, wherein the humidity proximate the first drug delivery device and the second drug delivery device is a minimum of about 10% and a maximum of about 95%.

21. The process of claim 1, wherein the temperature proximate the first drug delivery device and the second drug delivery device is a minimum of about 120° C. and a maximum of about 210° C.

22. The process of claim 1, wherein the temperature proximate the first drug delivery device differs from the temperature proximate the second drug delivery device by a maximum of about 25° C.

23. A process for making drug delivery devices, the process comprising the steps of:
(a) providing a therapeutically active agent in a first part and a second part;
(b) providing uncured poly(vinyl alcohol) in a first portion and a second portion;
(c) curing the first portion of poly(vinyl alcohol) and second portion of poly(vinyl alcohol), wherein the first portion and the second portion are separated by a predetermined distance for a time period, wherein the humidity proximate the first portion and the second portion varies by a maximum of 30% points relative humidity; and
(d) combining the first portion of poly(vinyl alcohol) and a second portion of poly(vinyl alcohol) with the respective first part and the second part in a respective first drug delivery device and a second drug delivery device.

24. The process of claim 23, wherein the first portion of poly(vinyl alcohol) and the second portion of poly(vinyl alcohol) are located relative to the respective first part and the second part in the respective first drug delivery device and the second drug delivery device to effect the rate of release of the first part and the second part from the drug delivery device.

25. The process of claim 23, wherein the first portion is mixed with the first part to form a matrix and the second portion is mixed with the second part to form a second matrix.

26. The process of claim 23, wherein the first part and the second part is formed into respective first drug core and second drug core and the first portion encapsulates at least a portion of the first drug core and the second portion encapsulates at least a portion of the second drug core.

27. The process of claim 26, wherein the first portion and the second portion encapsulates the entire first drug core and the entire second drug core, respectively.

28. The process of claim 26, wherein the first drug core and the second drug core are at least partly covered with an impermeable polymer material.

29. The process of claim 28, wherein the first portion and second portion form an inner covering and the impermeable polymer material form an outer coating.

30. The process of claim 23, wherein the step of combining occurs after the step of curing.

31. The process of claim 23, wherein the step of combining occurs before the step of curing.

32. The process of claim 26, wherein the step of providing further comprises providing a respective first drug core from the first portion and a second drug core from the second portion and further define providing a respective first cup and second cup that are impermeable to the passage of the therapeutically active agent and define respective first internal compartment and second internal compartment that are sized and configured to receive the first drug core and the second drug core respectively, the first unitary cup and the second unitary cup each define respective first opening and second opening;
wherein the step of providing a portion provides a respective first cover made from the first portion and second cover made from the second portion; and
wherein the step of combining further comprises placing the first cover in a covering relationship to the first opening and placing the second cover in a covering relationship to the second opening.

33. The process of claim 32, wherein the step of combining occurs after the step of curing.

34. The process of claim 23, wherein the first portion and the second portion, when cured, form a barrier through which the therapeutically active agent in each of the respective first drug delivery device and second drug delivery device passes into the eye of the patient.

35. The process of claim 23, wherein the first portion and the second portion, when cured, are positioned relative to the therapeutically active agent in each of the first drug delivery device and the second drug delivery device to effect the rate of release of therapeutically active agent from each of the first drug delivery device and second drug delivery device.

36. The process of claim 23, wherein the rate of release of therapeutically active agent from the first drug delivery device is lower than the rate of release of therapeutically active agent from the second drug delivery device by a maximum of about 50% based upon the rate of release of the second drug delivery device.

37. The process of claim 23, wherein the predetermined distance is a minimum of about 30 cm.

38. The process of claim 23, wherein the time period is a minimum of about 15 minutes and a maximum of about 24 hours.

39. The process of claim 23, wherein, wherein the therapeutically active agent is a hydrophobic agent.

40. The process of claim 23, wherein the therapeutically active agent is selected from the group comprising anesthetics, analgesics, antibiotics, cell transport/mobility impending agents, antiglaucoma drugs, carbonic anhydrase inhibitors, neuroprotectants, antibacterials, anti-fungal agents, anti-viral agents, protease inhibitors, anti-cytomegalovirus agents, antiallergenics, anti-inflammatories, decongestants, miotics, anti-cholinesterases, mydriatics, sympathomimetics, vasoconstrictors, vasodilators, anticlotting agents, antidiabetic agents, aldose reductase inhibitors, anti-cancer agents, hormones, peptides, nucleic acids, saccharides, lipids, glycolipids, glycoproteins, endocrine hormones, growth hormones, heat shock proteins, immunological response modifiers, cyclosporins, interferons (including [agr], [bgr], and [ggr] interferons), cytokines, antineogenesis proteins, monoclonal antibodies, tumor necrosis factor inhibitors, nulceic acids and mixtures thereof.

41. The process of claim 23, wherein the therapeutically active agent is present in an effective amount to treat glaucoma, proliferative vitreoretinopathy, diabetic retinopathy, uveitis, keratitis, cytomegalovirus retinitis, herpes simplex viral or adenoviral infections.

42. The process of claim 23, wherein the therapeutically active agent is selected from the group comprising colchicine, vincristine, cytochalasin B, timolol, betaxolol, atenolol, acetazolamide, methazolamide, dichlorphenamide, diamox, nimodipine, tetracycline, chlortetracycline, bacitracin, neomycin, polymyxin, gramicidin, oxytetracycline, chloramphenicol, gentamycin, and erythromycin; antibacterials such as sulfonamides, sulfacetamide, sulfamethizole sulfisoxazole, fluconazole, nitrofurazone, amphotericine B, ketoconazole, trifluorothymidine, acyclovir, ganciclovir, DDI, AZT, foscamet, vidarabine, trifluorouridine, idoxuridine, ribavirin, methapyriline, chlorpheniramine, pyrilamine, prophenpyridamine, hydrocortisone, dexamethasone, fluocinolone, prednisone, prednisolone, methylprednisolone, fluorometholone, betamethasone, triamcinolone, phenylephrine, naphazoline, tetrahydrazoline, pilocarpine, carbachol, di-isopropyl fluorophosphate, phospholine iodine, and demecarium bromide, atropine sulfate, cyclopentolate, homatropine, scopolamine, tropicamide, eucatropine, epinephrine, heparin, antifibrinogen, fibrinolysin, acetohexamide, chlorpropamide, glipizide, glyburide, tolazamide, tolbutamide, insulin, 5-fluorouracil, adriamycin, asparaginase, azacitidine, azathioprine, bleomycin, busulfan, carboplatin, carmustine, chlorambucil, cisplatin, cyclophosphamide, cyclosporine, cytarabine, dacarbazine, dactinomycin, daunorubicin, doxorubicin, estramustine, etoposide, etretinate, filgrastin, floxuridine, fludarabine, fluorouracil, fluoxymesterone, flutamide, goserelin, hydroxyurea, ifosfamide, leuprolide, levamisole, lomustine, nitrogen mustard, melphalan, mercaptopurine, methotrexate, mitomycin, mitotane, pentostatin, pipobroman, plicamycin, procarbazine, sargramostin, streptozocin, tamoxifen, taxol, teniposide, thioguanine, uracil, mustard, vinblastine, vincristine, vindesine, insulin-related growth factor, interleukin-2, tacrolimus, tumor necrosis factor, pentostatin, thymopentin, transforming factor beta-2, erythropoietin, anticlotting activase, brain nerve growth factor (BNGF), celiary nerve growth factor (CNGF), vascular endothelial growth factor (VEGF), thalidomide and mixtures thereof.

43. The process of claim 23, wherein the first drug delivery device and the second drug delivery device each have a maximum volume of 26 mm³.

44. The process of claim 23, wherein the first drug delivery device and the second drug delivery device each has a maximum mass of 50 mg.

45. The process of claim 23, wherein the therapeutically active agent comprises a minimum of about 10 wt. % and a maximum of about 95 wt. % of the total mass of the first drug delivery device and the second drug delivery device.

46. The process of claim 23, wherein the humidity proximate the first drug delivery device differs from the humidity proximate the second drug delivery device by a maximum of about 30% points relative humidity.

47. The process of claim 23, wherein the humidity proximate the first drug delivery device and the second drug delivery device is a minimum of about 10% and a maximum of about 95%.

48. The process of claim 23, wherein the temperature proximate the first drug delivery device and the second drug delivery device is a minimum of about 120° C. and a maximum of about 210° C.

49. The process of claim 23, wherein the temperature proximate the first drug delivery device differs from the temperature proximate the second drug delivery device by a maximum of about 25° C.

50. A process for making a plurality of drug delivery devices, the process comprising the steps of:
(a) providing a plurality of amounts of therapeutically active agent
(b) providing a plurality of portions of poly(vinyl alcohol);
(c) curing the plurality of portions that are separated by a predetermined distance for a time period, wherein the humidity proximate any one of the plurality of portions vary from any other of the plurality of portions by a maximum of 30% points relative humidity; and
(d) combining the plurality of amounts of therapeutically active agent with the plurality of portions of poly(vinyl alcohol) to form a plurality of drug delivery devices.

51. The process of claim 50, wherein each of the plurality of portions of poly(vinyl alcohol) are located relative to each of corresponding plurality of amounts of therapeutically active agent in each of corresponding plurality of drug delivery devices to effect the rate of release of therapeutically active agent from each of the plurality of drug delivery devices.

52. The process of claim 50, wherein each of the plurality of portions are mixed with each of corresponding plurality of amounts to form a corresponding plurality of matrices.

53. The process of claim 50, wherein the plurality of amounts are formed into a plurality of drug cores and the corresponding plurality of portions cover at least a portion of each of the plurality of drug cores.

54. The process of claim 53, wherein the plurality of portions each cover corresponding plurality of drug cores.

55. The process of claim 53, wherein the plurality of cores are in part covered with an impermeable polymer material.

56. The process of claim 55, wherein the plurality of portions form an inner covering over corresponding plurality of drug cores and the impermeable polymer material form an outer coating on each of the plurality of drug cores.

57. The process of claim 50, wherein the step of combining occurs after the step of curing.

58. The process of claim 50, wherein the step of combining occurs before the step of curing.

59. The process of claim 50, wherein the step of providing a plurality of amounts comprises providing a plurality of drug cores from the plurality of amounts, the plurality of drug cores are placed inside of a corresponding plurality of compartments that are defined by a plurality of cups, the plurality of cups are impermeable to the passage of the therapeutically active agent, the plurality of cups further define a corresponding plurality of openings;
wherein the step of providing a plurality of portions comprises providing a plurality of covers made from the plurality of portions; and
wherein the step of combining comprises placing the plurality of covers in a covering relationship to the first opening and placing the second cover in a covering relationship to the second opening.

60. The process of claim 59, wherein the step of combining occurs after the step of curing.

61. The process of claim 60, wherein the plurality of portions, when cured, form a barrier through which the therapeutically active agent in each of the plurality of drug delivery devices pass into the eye of the patient.

62. The process of claim 50, wherein the plurality of portions, when cured, are positioned relative to the therapeutically active agent in the plurality of drug delivery devices to effect the rate of release of therapeutically active agents from the plurality of drug delivery devices.

63. The process of claim 50, wherein the rate of release of therapeutically active agent from any one of the plurality of drug delivery devices is lower than the rate of release of another of the plurality of drug delivery devices by a maximum of about 50% based upon the rate of release of the second drug delivery device.

64. The process of claim 50, wherein the predetermined distance is a minimum of about 30 cm.

65. The process of claim 50, wherein the time period is a minimum of about 15 minutes and a maximum of about 24 hours.

66. The process of claim 50, wherein the therapeutically active agent is a hydrophobic agent.

67. The process of claim 50, wherein the therapeutically active agent is selected from the group comprising anesthetics, analgesics, antibiotics, cell transport/mobility impending agents, antiglaucoma drugs, carbonic anhydrase inhibitors, neuroprotectants, antibacterials, anti-fungal agents, anti-viral agents, protease inhibitors, anti-cytomegalovirus agents, antiallergenics, anti-inflammatories, decongestants, miotics, anti-cholinesterases, mydriatics, sympathomimetics, vasoconstrictors, vasodilators, anticlotting agents, antidiabetic agents, aldose reductase inhibitors, anti-cancer agents, hormones, peptides, nucleic acids, saccharides, lipids, glycolipids, glycoproteins, endocrine hormones, growth hormones, heat shock proteins, immunological response modifiers, cyclosporins, interferons (including [agr], [bgr], and [ggr] interferons), cytokines, antineogenesis proteins, monoclonal antibodies, tumor necrosis factor inhibitors, nulceic acids and mixtures thereof.

68. The process of claim 50, wherein the therapeutically active agent is present in an effective amount to treat glaucoma, proliferative vitreoretinopathy, diabetic retinopathy, uveitis, keratitis, cytomegalovirus retinitis, herpes simplex viral or adenoviral infections.

69. The process of claim 50, wherein the therapeutically active agent is selected from the group comprising colchicine, vincristine, cytochalasin B, timolol, betaxolol, atenolol, acetazolamide, methazolamide, dichlorphenamide, diamox, nimodipine, tetracycline, chlortetracycline, bacitracin, neomycin, polymyxin, gramicidin, oxytetracycline, chloramphenicol, gentamycin, and erythromycin; antibacterials such as sulfonamides, sulfacetamide, sulfamethizole sulfisoxazole, fluconazole, nitrofurazone, amphotericine B, ketoconazole, trifluorothymidine, acyclovir, ganciclovir, DDI, AZT, foscamet, vidarabine, trifluorouridine, idoxuridine, ribavirin, methapyriline, chlorpheniramine, pyrilamine, prophenpyridamine, hydrocortisone, dexamethasone, fluocinolone, prednisone, prednisolone, methylprednisolone, fluorometholone, betamethasone, triamcinolone, phenylephrine, naphazoline, tetrahydrazoline, pilocarpine, carbachol, di-isopropyl fluorophosphate, phospholine iodine, and demecarium bromide, atropine sulfate, cyclopentolate, homatropine, scopolamine, tropicamide, eucatropine, epinephrine, heparin, antifibrinogen, fibrinolysin, acetohexamide, chlorpropamide, glipizide, glyburide, tolazamide, tolbutamide, insulin, 5-fluorouracil, adriamycin, asparaginase, azacitidine, azathioprine, bleomycin, busulfan, carboplatin, carmustine, chlorambucil, cisplatin, cyclophosphamide, cyclosporine, cytarabine, dacarbazine, dactinomycin, daunorubicin, doxorubicin, estramustine, etoposide, etretinate, filgrastin, floxuridine, fludarabine, fluorouracil, fluoxymesterone, flutamide, goserelin, hydroxyurea, ifosfamide, leuprolide, levamisole, lomustine, nitrogen mustard, melphalan, mercaptopurine, methotrexate, mitomycin, mitotane, pentostatin, pipobroman, plicamycin, procarbazine, sargramostin, streptozocin, tamoxifen, taxol, teniposide, thioguanine, uracil, mustard, vinblastine, vincristine, vindesine, insulin-related growth factor, interleukin-2, tacrolimus, tumor necrosis factor, pentostatin, thymopentin, transforming factor beta-2, erythropoietin, anticlotting activase, brain nerve growth factor (BNGF), celiary nerve growth factor (CNGF), vascular endothelial growth factor (VEGF), thalidomide and mixtures thereof.

70. The process of claim 50, wherein each of the plurality of drug delivery devices has a maximum volume of 26 mm³.

71. The process of claim 50, wherein each of the plurality of drug delivery devices has a maximum mass of 50 mg.

72. The process of claim 50, wherein the therapeutically active agent comprises a minimum of about 10 wt. % and a maximum of about 95 wt. % of the total mass of the first drug delivery device and the second drug delivery device.

73. The process of claim 50, wherein the humidity proximate any one of the plurality of drug delivery devices differ from the humidity proximate another of the plurality of drug delivery devices by a maximum of about 25% points relative humidity.

74. The process of claim 50, wherein the average humidity proximate each of the plurality of drug delivery devices is a minimum of about 10% and a maximum of about 90%.

75. The process of claim 50, wherein the average temperature proximate each of the plurality of drug delivery devices is a minimum of about 120° C. and a maximum of about 210° C.

76. The process of claim 50, wherein the temperature proximate any one of the plurality of drug delivery devices differ from the temperature proximate another of the plurality of drug delivery devices by a maximum of about 25° C.

77. An inventory of devices comprising a plurality of drug delivery devices made according to the process of claim 50.
